(19) United States
(12) Patent Application Publication
(10) Pub. No.: US 2003/0125233 A1
(43) Pub. Date: Jul. 3, 2003

(54) Use of urokinase inhibitors for the treatment and/or prevention of pulmonary hypertension and/or cardiac remodelling

(76) Inventors: Peter Carmeliet, Landen (BE); Desire Collen, Winksele (BE); Stephane Heymans, Heverlee (BE); Marcel Levi, Amsterdam (NL)

Correspondence Address: Elie H Gendloff, Amster Rothstein & Ebenstein, 90 Park Avenue, New York, NY, 10016, US

(21) Appl. No.: 10/312,453

(22) PCT Filed: Jun. 27, 2001
(86) PCT No.: PCT/EP01/07312

(30) Foreign Application Priority Data
Jun. 27, 2000 (EP) 002202228.3

(51) Int. Cl. A61K 31/00

(52) U.S. Cl. 514/1

(57) ABSTRACT

The present invention relates to methods of treatment and/or prevention of pulmonary hypertension and to methods of treatment and/or prevention of cardiac remodelling and more specifically to cardiac remodelling induced by systemic hypertension in a mammal, particularly a human being. In particular, the invention shows a novel, negative role for urokinase-type plasminogen activator in the pathogenesis of cardiac remodelling, leading to subsequent cardiac dysfunction, and in the pathogenesis of pulmonary hypertension usually complicated by subsequent right ventricular hypertrophy. Consequently, the use of selective inhibitors of u-PA activity can be of benefit for treatment of patients suffering from pulmonary hypertension and/or cardiac remodelling.

### FIELD OF THE INVENTION

The present invention relates to methods of treatment and/or prevention of pulmonary hypertension and to methods of treatment and/or prevention of cardiac remodelling and more specifically to cardiac remodelling induced by systemic hypertension in a mammal, particularly a human being. In particular, the invention shows a novel, negative role for urokinase-type plasminogen activator in the pathogenesis of cardiac remodelling, leading to subsequent cardiac dysfunction, and in the pathogenesis of pulmonary hypertension usually complicated by subsequent right ventricular hypertrophy. Consequently, the use of selective inhibitors of u-PA activity can be of benefit for treatment of patients suffering from pulmonary hypertension and/or cardiac remodelling.

### BACKGROUND OF THE INVENTION

More than 10 percent of Western population suffers from severe hypertension. In young patients, severe hypertension first results in pronounced hypertrophic cardiomyopathy. The magnitude of left ventricular (LV) hypertophy in these patients is a strong and independent predictor of the risk of sudden death and indeed more than 20 percent of young patients with severe hypertrophic cardiomyopathy die of sudden death before the age of 40 years (Spirito et al., (2000), N. Engl. J. Med., 342, 1778). In older patients, long-term hypertension may end in congestive heart failure. The prognosis of progressive heart failure is poor: more than 50 percent of these patients die within one year (Ju et al. (1996) Can. J. Cardiol. 12, 1259). Left ventricular (LV) hypertrophy initially occurs as an adaptation of the heart to an increased systolic wall stress. Major increase in myocyte volume and collagen deposition around the larger coronary vessels and in the interstitium characterizes the initial stage of hypertension and results in hypertrophic cardiomyopathy. When hypertrophy becomes chronic, myocytes degenerate, die and are replaced by matrix-producing fibroblasts. Myocyte necrosis and related fibrosis result in increased myocardial stiffness, systolic dysfunction, and ultimately in progressive cardiac failure (Jaffe et al. (1997), Adv. Exp. Med. Biol. 430, 257). Increased matrix metalloproteinase (MMP) and plasminogen activator (PA) activity have been demonstrated during LV remodeling after acute myocardial infarction. Pharmacological proteinase inhibition reduces LV dilatation after acute myocardial infarction and prevents cardiac dysfunction in a pacing model of LV failure. Direct evidence for a role of MMP proteinases in hypertrophy of the myocardial cell and in progression to cardiac failure during severe hypertension is, however, lacking. In addition, the role of the plasminogen system, comprising urokinase-type plasminogen activator (u-PA), tissue-type PA (t-PA) and their PA inhibitor-1 (PAI-1), in myocyte hypertrophy and cardiac failure during LV hypertension remains completely undetermined.

The present invention shows that deficiency of u-PA or an uPA-inhibition by adenoviral gene transfer of PAI-1 impairs LV hypertrophy and prevents cardiac dysfunction during pressure-overload induced hypertension indicating a central, novel role of u-PA-mediated proteolytic activity in hypertensive cardiomyopathy.

A specific form of hypertension in the lung is pulmonary hypertension. Although the exact cause of pulmonary hypertension is not known it is believed that hypoxia is implicated. Hypoxia can affect the function of blood vessels in many ways. It is one of the most potent stimuli for the formation of new blood vessels (angiogenesis) and it mediates vasodilatation of vessels, thereby improving tissue perfusion¹. Lung vessels, however, react to acute hypoxia with constriction rather than dilatation, in part via upregulation of vaso-active substances²⁻⁵. Furthermore, hypoxia causes pulmonary vascular cells to proliferate in contrast to the usual growth-suppressive effect of hypoxia on many other cell types²⁻⁷. Upon prolonged hypoxia, pulmonary vessels undergo significant structural changes involving medial thickening of alveolar duct and terminal bronchiolar arteries due to smooth muscle cell accumulation and matrix deposition, and extension of a muscular wall in intra-acinar vessels⁸⁻⁹. The latter may result from increased proliferation of smooth muscle cells to more distal uncovered vessels, or from recruitment and differentiation of pulmonary fibroblasts or pericytes to contractile smooth muscle cells. Another characteristic feature of vascular remodeling during pulmonary hypertension involves rarefaction of pulmonary arteries¹⁰. Although poorly understood, rarefaction presumably reflects an adaptive structural response to prune insufficiently perfused ‘ghost’ arterioles, formed following severe microvascular constriction¹¹. As a consequence, pulmonary hypertension and right ventricular hypertrophy may develop, ultimately progressing to right heart failure. This constitutes a major causes of cardiopulmonary morbidity and mortality, e.g. in patients with chronic obstructive lung disease, for which few medical treatment exists. The precise molecular mechanisms, which play a role in the pathogenesis of pulmonary hypertension and the structural changes in the pulmonary vasculature, are only partly known¹². Pulmonary vasoconstriction appears to be of importance and both endothelin and angiotensin have been implicated as important mediators^{13,14}. In fact, either inhibition of endothelin or endothelin receptor blockade or inhibition of angiotensin was shown to prevent hypoxia-induced pulmonary hypertension and vascular remodeling in rats^{15,16}. In contrast, the observation that mice deficient in eNOS were found to develop excessive pulmonary hypertension¹⁷, whereas adenoviral NOS gene transfer prevented hypoxia-induced pulmonary hypertension¹⁸ point to an important role of NO in the prevention of hypoxia-induced pulmonary vasoconstriction. In addition, several vascular cell mitogens, such as heparin-binding epidermal growth factor (HG-EGF)¹⁹, vascular endothelial cell growth factor (VEGF)²⁰, and platelet-derived growth factor (PDGF)²¹ appear to be implicated in the pathogenesis of pulmonary hypertension. Lastly, activation of proteases, in particular elastase, may be essential for extracellular matrix degradation associated with pulmonary vascular remodeling. It has been clearly shown that inhibition of elastase protects against pulmonary hypertension in rats²². Elastase was also shown to be able to induce the release of growth factors (such as basic fibroblast growth factor (b-FGF)) from the extracellular matrix, which may further contribute to pulmonary artery smooth muscle proliferation²³.

In the present invention the role of the plasminogen system in the pathogenesis of pulmonary hypertension and right ventricular remodeling was studied. A surprising role for urokinase was found in hypoxia-induced pulmonary vascular remodeling and subsequent right ventricular hypertrophy. These findings can have important consequences for preventive or therapeutic strategies in patients with (evolving) pulmonary hypertension.

### AIMS OF THE INVENTION

The present invention aims at providing therapeutics in order to improve health of patients suffering from the consequences of systemic hypertension and of patients suffering from pulmonary hypertension. In particular, the invention aims at providing the usage of urokinase inhibitors for the manufacture of a medicament, in order to treat patients suffering from cardiac remodelling and more particularly cardiac remodelling induced by systemic hypertension. The present invention also aims at using urokinase inhibitors for the manufacture of a medicament for the prevention and/or treatment of pulmonary hypertension and the prevention of right heart failure that occurs as a consequence of pulmonary hypertension. The present invention further aims at providing a pharmaceutical composition for the before mentioned treatments. Another aim of the invention is the use of plasminogen activator inhibitors (PAI) and functional fragments thereof for gene therapy of pulmonary hypertension and/or cardiac remodelling.

### DETAILED DESCRIPTION OF THE INVENTION

In the present invention a novel role for urokinase-type plasminogen activator (u-PA) was found in the pathogenesis of cardiac remodeling, leading to subsequent cardiac dysfunction, due to systemic hypertension and also in the pathogenesis of pulmonary hypertension, usually complicated by subsequent right ventricular hypertrophy³⁴.

In a mouse model for high pressure-induced left ventricular hypertrophy it was unexpectedly found that u-PA^{-/-} mice are significantly protected against cardiac remodeling and subsequent cardiac failure. Specifically, the present invention shows that deficiency of u-PA impairs myocyte hypertrophy, reduces myocyte loss and interstitial fibrosis, thereby preserving left ventricular (LV) contractility and function. In concordance, the results indicate that u-PA inhibition by adenoviral gene transfer of PAI-1 impairs said LV hypertrophy and prevents cardiac dysfunction during pressure-overload induced hypertension, confirming a central role of u-PA—mediated proteolytic activity in hypertensive cardiomyopathy.

Furthermore, in a mouse model for pulmonary hypertension it was surprisingly found that u-PA^{-/-} and plasminogen^{-/-} mice were resistant to hypoxia-induced anatomical and functional changes, whereas t-PA^{-/-} mice responded to hypoxia in an identical manner as wild type mice. Hypoxic u-PAR^{-/-} mice showed an intermediate response, which is significantly lower than wild type mice. The present invention teaches that u-PA^{-/-} and plg^{-/-} mice do not show hypoxia-induced fragmentation of the elastic membrane and subsequent pulmonary vascular remodeling. Furthermore, the development of right ventricular hypertrophy, a direct consequence of an enhanced pulmonary artery pressure, is not observed in u-PA^{-/-} and plg^{-/31} mice. Therefore, the present invention is relevant for the management of hypoxic pulmonary hypertension and right ventricular hypertrophy in patients since the murine hypoxia model has generally been accepted as a model for human hypoxic disease^{10,40}. Consequently, the use of selective inhibitors of u-PA activity, can be of benefit for patients with pulmonary vascular disease due to chronic hypoxia. At present, there is no specific therapy for this condition and the occurrence of pulmonary vascular disease and secondary right ventricular hypertrophy and subsequent right heart failure is associated with considerable morbidity and mortality. Urokinase (urinary-type plasminogen activator or u-PA; International Union of Biochemistry classification number: EC 3.4.21.31) is a proteolytic enzyme which is highly specific for a single peptide bond in plasminogen. Cleavage of this bond by urokinase (“plasminogen activation”) results in formation of the potent general protease plasmin. Cellular invasiveness initiated by urokinase is central to a wide variety of normal and disease-state physiological processes (reviewed in: Blasi et al. J. Cell Biol. 104:801, 1987; Dan et al. Adv. Cancer Res. 44:139, 1985; Littlefield et al. Ann. N. Y. Acad. Sci. 622:167, 1991; Testa et al. Cancer Metast. Rev. 9:353, 1990). Inhibitors of urokinase therefore have been suggested for for mechanism-based anti-angiogenic, anti-arthritic, anti-inflammatory, anti-invasive, anti-metastatic, anti-osteoporotic, anti-retinopathic (for angiogenesis-dependent retinopathies), contraceptive, and tumoristatic activities but said inhibitors have never been suggested for the treatment and/or prevention of cardiac remodeling and/or for the treatment and/or prevention of pulmonary hypertension.

Beneficial effects of urokinase inhibitors have been reported in the prior art using anti-urokinase monoclonal antibodies and certain other known urokinase inhibitors. For instance, anti-urokinase monoclonal antibodies have been reported to block tumor cell invasiveness in vitro (Hollas et al. Cancer Res. 51:3690, 1991; Meissauer et al. Exp. Cell Res. 192:453, 1991), tumor metastasis and invasion in vivo (Ossowski, Cell Biol. 107:2437-2445, 1988; Ossowski et al. Cancer Res. 51:274, 1991), and angiogenesis in vivo (Jerdan et al. J. Cell Biol. 115:402a, 1991). For example, amiloride, a known urokinase inhibitor of only moderate potency, has been reported to inhibit tumor metastasis in vivo (Kellen et al. Anticancer Res. 8:1373, 1988) and angiogenesis/capillary network formation in vitro (Alliegro et al. J. Cell Biol. 115:402a, 1991).

Several patents describe more powerful urokinase inhibitors. Examples are described in U.S. Pat. No. 5,340,833, U.S. Pat. No. 5,550,213, U.S. Pat. No. 6,207,701, WO 2001014324, EP 1044967, U.S. Pat. No. 6,093,731, CH 689611, WO 2000006154, WO 2000005245, WO 2000005214, U.S. Pat. No. 5,952,307, WO 9940088, WO 9920608, WO 9905096, WO 9811089, EP 568289 and WO 2001044172. The plasminogen system (Libby (1995) Circulation, 11, 2844) consists of an inactive zymogen plasminogen (Plg) which can be converted to plasmin by two types of plasminogen activators (PAs), tissue-type PA (t-PA), generally believed to be mainly associated with fibrinolysis due to its fibrin specificity, and urokinase-type PA (u-PA), which binds to its receptor u-PAR and is implicated in cell migration and tissue remodelling. The system is controlled by plasminogen activator inhibitor-1 (PAI-1), the main physiological inhibitor of u-PA and t-PA, as well as by alfa2-antiplasmin (directly inhibiting plasmin).

The MMP system is a growing family of Zn²⁺ and Ca²⁺-dependent proteinases able to degrade most extracellular matrix proteins (Mignatti et al. (1996) Enzyme Protein, 49, 117). Based on substrate specificity and structural features, different groups can be distinguished. Collagenases (MMP-1, -8, -13 and -18) mostly degrade fibrillar collagens while gelatinases (MMP-2 and MMP-9) mainly degrade collagen type IV and denatured collagens. Stromelysin-1 and -2 (MMP-3 and MMP-10) and matrilysin (MMP-7) have a broad substrate specificity including proteoglycan core proteins, laminin, fibronectin, gelatin, non-helical collagens and elastin, whereas stromelysin-3 (MMP-11) does not degrade any of the major extracellular matrix components but targets serine proteinase inhibitors (serpins) like alfa-1 proteinase inhibitor. Metalloelastase (MMP-12) primarily degrades elastin, and the membrane-type metalloproteinases (MT-MMPs; MMP-14-17) have an additional transmembrane domain anchoring them to the cell surface. The system is controlled at several levels: (i) transcriptional control by growth factors and cytokines, (ii) activation of the inactive zymogens (pro-MMPs); u-PA-generated plasmin is a likely pathological activator of several pro-MMPs [16]; (iii) tissue- and substrate-specific inhibition of the active enzymes by tissue inhibitors of MMPs (TIMPs) of which 4 members are known to date. As used herein ‘hypertension’ comprises systemic hypertension, essential hypertension, malignant hypertension, renal hypertension and pulmonary hypertension.

Systemic hypertension, also called high blood pressure, is a condition in which the blood pressure in either arteries or veins is abnormally high. Blood pressure is defined as the force exerted by the blood against the walls of the blood vessels. Normally, the pumping of the heart creates a rhythmic pulsing of blood along and against the walls of the blood vessels, which are flexible enough to dilate or contract and thus keep the pressure constant. Most physicians consider the systemic blood pressure of a healthy adult to be in the neighbourhood of 120/80—i.e., equivalent to the pressure exerted by a column of mercury 120 mm high during contraction of the heart (systole) and 80 mm high during relaxation (diastole). Sometimes, however, for a variety of reasons, the blood vessels may lose their flexibility, or the muscles surrounding them may force them to contract. As a result, the heart must pump more forcefully to move the same amount of blood through the narrowed vessels into the capillaries, thereby increasing the blood pressure. Regardless of the mechanism, a sustained elevation of blood pressure for a period of time has been shown to result in significant cardiovascular damage throughout the body, e.g., congestive heart failure, coronary artery disease, stroke and progressive renal failure. Congestive heart failure frequently constitutes an end-stage complication of cardiac overload due to systemic hypertension or cardiac valve dysfunctions but may also result from acute or chronic ischemic heart disease and idiopathic cardiomyopathies (Battegay (1995) J. Mol. Med. 73, 333). Patients suffering from systemic hypertension or aortic valve dysfunction can benefit from adequate drug treatment or valve replacements, but hypertrophy and heart failure may become irreversible (Golia et al. (1997) Int. J. Cardiol. 60, 81).

Systemic hypertension is generally classified by cause either as essential (of unknown origin) or as secondary (the result of a specific disease, disorder, or other condition). Secondary hypertension may result from a wide range of causes. For example, renal hypertension affects the entire systemic circulation and arises from hypertension within the renal arteries, which branch from the aorta to supply blood to the kidneys. Hypertension may also result from the excess hormones that are secreted during abnormal functioning of the outer substance, or cortex, of the adrenal glands (Cushing's syndrome; aldosteronism); from the excess hormones resulting from pheochromocytoma, which is a tumour of the inner substance (medulla) of the adrenal glands; or from the excess hormones secreted by pituitary tumours. Other causes of secondary hypertension are coarctation—localized narrowing—of the aorta, pregnancy, and the use of oral contraceptives. In all secondary cases, the hypertension is relieved by treating the underlying condition or cause. By far the most common form of hypertension (90 percent of cases) is essential, or idiopathic, hypertension. Although no specific cause can be determined in such cases, studies have pointed out several contributing factors. Included among these are a family history of hypertension, obesity, high salt intake, smoking, and most importantly, emotional and physical stress.

In its milder forms, essential hypertension is usually treated with a self-help regimen that includes a no-salt diet and perhaps a weight-reducing diet, a decrease in or cessation of smoking, mild exercise, and the avoidance of or more successful coping with stressful situations. If a self-help program does not help lower the patient's blood pressure, the physician will usually prescribe diuretics or sympathetic-nerve blockers. The nerve blockers generally act by decreasing heart output and peripheral resistance to blood flow. Beta blockers are the most commonly used of these drugs and include metoprolol, nadolol, and propranolol. More severe hypertension often requires the use of drugs called vasodilators, which dilate the arteries, thus lowering the blood pressure. Oral vasodilators, which include hydralazine and minoxidil, are often used in conjunction with a diuretic and a sympathetic nerve blocker to inhibit the body's natural tendency to increase fluid retention and increase blood flow in response to the arterial dilation. Severe and immediately life-threatening hypertension, either secondary or essential, is called malignant hypertension and usually requires hospitalization and acute medical care. Treatment includes the intravenous administration of vasodilators such as diazoxide. “Pulmonary hypertension” is a specific condition of hypertension in the lung and relates to arterial hypertension, capillary hypertension or venous-hypertension in the lung. Suitably, the term “pulmonary hypertension” relates to pulmonary arterial hypertension. Furthermore it will be understood that pulmonary arterial hypertension relates to—but is not restricted to—both primary arterial hypertension and to pulmonary arterial hypertension occurring secondary to pulmonary diseases such as chronic bronchitis, emphysema, kyphoscoliosis and conditions such as chronic mountain sickness. Pulmonary hypertension is a serious medical condition that may lead to right ventricular hypertrophy, failure and death. When used herein the term “right heart failure” relates to disorders such as cor pulmonale and congenital abnormalities of the heart. It will be appreciated that cor pulmonale often occurs secondary to certain lung diseases such as chronic bronchitis and emphysema. Congenital abnormalities of the heart include disorders, such as atrial septal defect, tetralogy of fallot, venticular septal defect and persistent ductus arteriosus.

A fundamental event in the progression of heart failure due to dilated cardiomyopathy is left ventricular (LV) “myocardial remodeling”. The term “cardiac remodeling” has been coined to describe the geometrical changes in size and shape of the heart ventricle and also involves changes on a cellular level due to remodeling of the interstitial matrix which can lead to processes comprising fibrosis, myocyte necrosis and myocyte hypertrophy. It is not exactly known what initiates the process of cardiac remodeling process. Slipping of myofilaments following destruction of connective tissue could be the initial event. In the prior art it is believed that matrix metalloproteinases (MMP) start this process and are therefore an important therapeutic target (Spinale et al. (2000) Cardiovasc. Res. 46, 225). In the present invention evidence is presented that in addition urokinase also plays a prominent role in cardiac remodeling. As a consequence of myofilament slipping, wall stress is increased, triggering deleterious adaptation process, such as:—intracardic angiotension II generation;—cardiac endothelin formation and release;—pro-apoptotic signals for cardiomyocytes;—hypertrophic signals for fibroblasts and cardiomyocytes. This cascade of events is not only observed in the process of remodeling during the progression of heart failure but also following myocardial infarction. Current therapeutic principles therefore are similar in both conditions:—reduction of wall stress (pharmacological or mechanical unloading of the heart);—blockade of angiotensin II generation or of AT1-receptors (ACE-inhibitors or AT1 antagonists);—blockade of endothelin receptors (ET(A)-blockers);—blockade of adrenergic receptors (preferably beta1-adrenergic receptor blockers). Better understanding of the molecular mechanisms of the remodeling process already has fueled the search for new therapeutic interventions (such as endothelin receptor blockers, aldosterone antagonists, MMP-inhibitors and growth hormone application in cardiac remodeling.

In particular the present invention provides the use of urokinase inhibitors or a pharmaceutically acceptable salt thereof, or a pharmaceutically acceptable solvate thereof for the manufacture of a medicament for the treatment and/or prophylaxis of disorders associated with pulmonary hypertension and/or the treatment and/or prophylaxis of disorders associated with cardiac remodeling and more specifically cardiac remodeling induced by essential hypertension.

In a specific embodiment a medicament comprising a combination between urokinase inhibitors and above described therapeutics, such as MMP-inhibitors, can be manufactured to prevent and/ or to treat cardiac remodeling and/or pulmonary hypertension.

The administration of a compound, here a urokinase inhibitor or a pharmaceutically acceptable salt thereof may be by way of oral, inhaled or parenteral administration, and preferably for the treatment and/or prevention of pulmonary hypertension by inhaled administration. The active compound may be administered alone or preferably formulated as a pharmaceutical composition.

An amount effective to treat the disorders hereinbefore described depends on the usual factors such as the nature and severity of the disorders being treated and the weight of the mammal. However, a unit dose will normally contain 0.01 to 50 mg for example 0.01 to 10 mg, or 0.05 to 2 mg of urokinase inhibitor or a pharmaceutically acceptable salt thereof. Unit doses will normally be administered once or more than once a day, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg. It is greatly preferred that the urokinase inhibitor or a pharmaceutically acceptable salt thereof is administered in the form of a unit-dose composition, such as a unit dose oral, parenteral, or preferably inhaled composition for the treatment and/or prevention of pulmonary hypertension.

Such compositions are prepared by admixture and are suitably adapted for oral, inhaled or parenteral administration, and as such may be in the form of tablets, capsules, oral liquid preparations, powders, granules, lozenges, reconstitutable powders, injectable and infusable solutions or suspensions or suppositories or aerosols.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well-known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

Oral formulations also include conventional sustained release formulations, such as tablets or granules having an enteric coating.

Preferably, compositions for use in the treatment and/or prevention of pulmonary hypertension are presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, for example between 1 and 5 microns, such as between 2 and 5 microns.

A favored inhaled dose will be in the range of 0.05 to 2 mg, for example 0.05 to 0.5 mg, 0.1 to 1 mg or 0.5 to 2 mg.

For parenteral administration, fluid unit dose forms are prepared containing a compound of the present invention and a sterile vehicle. The active compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and sterilised by exposure to ethylene oxide before suspending in the sterile vehicle. Advantageously, a surfactant or wetting agent is included in the composition to facilitate uniform distribution of the active compound. Where appropriate, small amounts of bronchodilators for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included.

As is common practice, the compositions will usually be accompanied by written or printed directions for use in the medical treatment concerned.

The present invention further provides a pharmaceutical composition for use in the treatment and/or prophylaxis of disorders associated with pulmonary hypertension and/or cardiac remodeling induced by systemic hypertension, which comprises a urokinase inhibitor and/or a combination as above described or a pharmaceutically acceptable salt thereof, or a pharmaceutically acceptable solvate thereof, and, if required, a pharmaceutically acceptable carrier thereof.

A particularly favored pharmaceutically acceptable composition for the treatment and/or prevention of pulmonary hypertension is an inhalation composition, suitably in unit dosage form. Such compositions may be prepared in the manner as hereinbefore described.

In another embodiment of the invention inhibitors of u-PA can be used for the treatment and/or prophylaxis of pulmonary hypertension and for the treatment and/or prophylaxis of cardiac remodelling which can occur as a complication of systemic hypertension. Examples of said inhibitors comprise plasminogen activator inhibitors, PAI-1 and PAI-2. As a possibility, but not limited to this, for the treatment of pulmonary hypertension, is to put the coding information of PAI-1 under control of a Hypoxia Response Element (HRE) (Semenza et al. (2000) Biochem. Pharmacol. 59, 47) in order to switch on the expression of PAI-1 only in hypoxic conditions. These genetic constructs can then be used in gene therapy. Gene therapy means the treatment by the delivery of therapeutic nucleic acids to patient's cells. This is extensively reviewed in Lever and Goodfellow 1995; Br. Med Bull., 51, 1-242; Culver 1995; Ledley, F. D. 1995. Hum. Gene Ther. 6, 1129. To achieve gene therapy there must be a method of delivering genes to the patient's cells and additional methods to ensure the effective production of any therapeutic genes. There are two general approaches to achieve gene delivery; these are non-viral delivery and virus-mediated gene delivery. As an example, but not limited to this, is the use of a virus-mediated gene delivery system with replication defective retroviruses to stably introduce genes into patient's cells. As a not limited possibility for the treatment of pulmonary hypertension, viral or non-viral delivery of therapeutic genes, can be administered in an inhalation composition.

The present invention will now be illustrated by reference to the following examples which set forth particularly advantageous embodiments. However, it should be noted that these embodiments are illustrative and cannot be construed as to restrict the invention in any way.

### EXAMPLES

1.1 Cardiac Hypertrophy in Pressure-overload Mice.

As shown in Table 2, transverse aortic banding (TAB) resulted in a large increase of LV pressure in wild type (WT) mice at 14 and 49 days. This severe hypertension resulted in 51% increase of LV/body weight ratio in WT mice, concordant with a significant increase in cross-sectional area of LV cardiomyocytes 14 days after TAB as compared to sham (Table 1). Myocardial fibrosis, secondary to reactive interstitial fibrosis and to repair of myocyte necrosis, was evident in WT mice 14 days after TAB and was most pronounced in the subendocardial areas. Collagen content (% Sirius red stained area per total area) increased by 95% in WT mice 14 days after TAB (Table 1).

1.2 Absence of u-PA Impairs Myocyte Hypertrophy and Cardiac Fibrosis.

Increase of LV pressure was similar in u-PA-deficient as compared to WT mice 14 and 49 days after TAB (Table2). However, the LV/body weight ratio increased significantly less in u-PA-deficient as compared to WT mice at 14 and 49 days after TAB (Table 1). In concordance, increase of cross-sectional area of LV cardiomyocytes after TAB was significantly less in u-PA-deficient mice. In addition, LV collagen content only minimally changed in u-PA-deficient mice after TAB as compared to sham-operated mice (Table 1).

1.3 Improved Cardiac Function in u-PA Deficient Mice.

Intraventricular pressure measurements were obtained in both wild type and u-PA-deficient mice at 14 and 49 days after TAB (Table 2). In WT mice, both contractility (measured as +dP/dtₘₐₓ) and relaxation (measured as −dP/dtₘᵢₙ) did not significantly change at 14 days, whereas contractility significantly decreased at 49 days after TAB as compared to sham-operated WT mice. In contrast, reduced hypertrophy and cardiac fibrosis in u-PA-deficient hearts resulted in a significant increase of contractility and relaxation at 14 and 49 days after TAB (Table 2).

In vivo M-mode echocardiograms were obtained in both wild type and u-PA-deficient mice at 14 days after TAB. As demonstrated in table 3, posterior and septal wall thickness increased in WT-banded mice as compared to sham, whereas wall thickness only minimally changed in u-PA-deficient mice after TAB. In both wild type and u-PA-deficient mice, LV internal diastolic diameter remained unchanged. However, LV internal systolic diameter was increased in WT mice after TAB as compared to sham. This resulted in a large decrease of fractional shortening in wild type mice, suggesting systolic dysfunction, whereas fractional shortening remained unchanged in u-PA-deficient mice.

1.4 PAI-1 Gene Transfer Reduces LV Hypertrophy and Improves LV Contractility.

In order to confirm the previous results and to investigate whether u-PA inhibition could reduce cardiac hypertrophy and thereby improve cardiac function, 1.2×10⁹ pfu of a replication-deficient adenovirus expressing human PAI-1 (AdPAI-1) or a control gene (AdRR5) was injected intravenously in WT mice three days before TAB. AdPAI-1 elevated plasma PAI-1 levels to 54±3 μg/ml within five days. AdPAI-1 reduced LV hypertrophy, as indicated by decreased LV/body weight ratio as compared to AdRR5-treated mice (Table 4). Impaired hypertrophy resulted in improved cardiac function, as demonstrated by an increased LV relaxation and contractility after PAI-1-gene transfer as compared to control mice (Table 4).

Thus adenoviral PAI-1-gene transfer reduced LV hypertrophy and improved LV contractility. Inhibition of the plasminogen system may therefore constitute a novel treatment strategy to impair cardiac hypertrophy secondary to severe systemic hypertension.

2.1 Absence of u-PA Protects Again Pulmonary Hypertension

Adult wild type mice that were exposed to 28 days of hypoxia showed a significant 1.8 to 2.7-fold rise in right ventricular pressure (table 5). The right ventricular systolic and diastolic pressure was 37 (±3.8) mmHg and 15 (±3.7) mmHg, respectively, as compared with right ventricular systolic and diastolic pressures of 21 (±3.2) and 5.5 (±1.0) mmHg in normoxic mice (p<0.01). In u-PA^{-/-} mice no such increase in right ventricular pressure was seen. In contrast, t-PA^{-/-} mice showed a rise in right ventricular pressure in response to hypoxia that was fully comparable with that of wild type mice. Mice with a deficiency in the u-PA receptor showed a partial but significant hypoxia-induced enhancement in right ventricular pressure. Plasminogen deficient mice had no significant increase in right ventricular pressure in response to hypoxia. Hypoxia did not affect mean arterial blood pressure in any of the groups (table 5).

Hypoxia resulted in an increase in hematocrit from 48.7 (±1.0)% to 61.1 (±0.9)%. This increase in hematocrit was identical in all genotypes studied (table 5).

2.2 Right Ventricular Weight

In wild type mice hypoxia caused a 1.7-fold increase in the right ventricle/left ventricle+septum ratio and a 1.8-fold increase in the right ventricle weight/body weight ratio. In accordance with the right ventricular pressure measurements, u-PA^{-/-} and plg^{-/-} mice did not show any increase in right ventricular weight, whereas t-PA deficient mice showed right ventricular hypertrophy that was comparable to wild type mice. Hypoxia caused a significant increase in right ventricular weight in u-PAR^{-/-} mice, however, again this increase was more modest as compared with the increase in wild type mice (RV/LV+S ratio in u-PAR-^{-/-} mice 42 (±6)% as compared with 51 (±5)% in wild type mice (p<0.05)).

In newborn mice a similar pattern was observed. Both the right ventricle/left ventricle+septum ratio and the right ventricle weight/body weight ratio were markedly increased in wild type mice (1.7 to 1.9-fold) and t-PA^{-/-} mice (1.6 to 2.0-fold) exposed to hypoxia. In contrast, u-PA^{-/-} mice did not show any significant increase in right ventricular hypertrophy in response to hypoxia. Total bodyweight was not different between hypoxic and normoxic mice of all different genotypes.

2.3 Pulmonary Vascular Remodelling

As shown in table 6, hypoxia induced mild vascular rarefaction in the lungs of wild type mice. In wild type mice exposed to hypoxia, a 29% reduction in non-muscularized vessels and a 22% reduction in partly or fully muscularized arterioles was observed (p<0.05). Both u-PA^{-/-} mice and plg^{-/-} deficient mice did not show such a reduction in vascular density in response to hypoxia. The hypoxia-induced rarefaction in t-PA^{-/-} mice was similar to that in wild type mice. U-PAR^{-/-} mice showed an intermediate reduction in the number of arteries per 100 alveoli.

The increase in smooth muscle cells within the distal arterial walls, as reflected by the increase in media thickness, followed a similar pattern. In wild type mice hypoxia caused an almost 2-fold increase in the ratio of media thickness over vascular diameter, which was similar in t-PA^{-/-} mice. Conversely, u-PA^{-/-} mice and plg^{-/-} mice did not show an increase in media thickness (table 6). In mice with a deficiency of the u-PA receptor a significant increase in media thickness was observed, however, to a lesser extent that the increase seen in wild type or t-PA^{-/-} mice. Interestingly, hypoxic wild type or t-PA^{-/-} mice showed a marked fragmentation of the elastic membrane, whereas this was not seen in u-PA^{-/-} or plg^{-/-} mice.

Pulmonary vascular remodeling in response to hypoxia was even somewhat more pronounced in newborn wild type mice but was again completely absent in u-PA^{-/-} mice. Both vascular density and media thickness in u-PA^{-/-} mice exposed to hypoxia were virtually unchanged as compared with normoxic controls, whereas t-PA^{-/-} mice showed signs of vascular remodeling that were identical to wild type mice.

There were no differences in pulmonary vascular density or media thickness between genotypes at normoxic conditions.

2.4 Histological Analysis of Right Ventricular Hypertrophy

Histological analysis revealed a hypoxia-induced increase in right ventricular cardiomyocyte size from 250 (±40) μm² to 340 (±68) μm² in wild type mice, which was not present in u-PA^{-/-} mice (table 7). Also, the almost 2-fold increase in collagen content of the right ventricular wall in hypoxic wild type mice was not seen in u-PA^{-/-} mice (table 7). Right ventricular remodeling upon hypoxia in wild type mice was associated with a small but not significant reduction in subendocardial capillary density (from 5200 (±210)/mm² to 4400 (±260)/mm²).

2.5 Expression and Zymographic Activity of Plasminogen Activators and MMP-9 in Lungs and Hearts

Immunostaining for u-PA revealed enhanced u-PA expression in lungs of hypoxic wild type mice, in particular located near vascular smooth muscle cells. Zymographic analysis showed a 1.8 (±0.3) -fold increase in u-PA activity in these lungs. In addition, there was increased MMP-9 expression (which might be seen as a candidate for u-PA-mediated plasmin formation)³⁰ related to macrophages, in particular around the pulmonary vasculature. There was no major difference in the expression or zymographic activity of plasminogen activators in hearts from hypoxic mice as compared with hearts from normoxic controls.

2.6 Use of Urokinase Inhibitors for the Treatment of Pulmonary Hypertension

Several urokinase inhibitors are currently being evaluated for the treatment of pulmonary hypertension in a murine model for pulmonary hypertension.

2.7 Use of Urokinase Inhibitors for the Treatment of Hypertensive Cardiomyopathy

Several urokinase inhibitors are currently being evaluated for the treatment of cardiac remodeling in a murine model for hypertensive cardiomyopathy.

Materials and Methods

Animals and experimental protocol

The experiments were approved by the Institutional Review Board and were conducted according to the guidelines for animal experiments of the NIH. Transgenic mice and appropriate wild type control mice were studied after being exposed to chronic hypoxia, as compared with normoxic conditions (controls). Experimental groups consisted of the following adult (6-8 weeks old) mice: (1) t-PA^{-/-} mice, (2) u-PA^{-/-} mice, (3) u-PAR^{-/-} mice, (4) plasminogen^{-/-} mice, and (5) wild type mice. In addition, newborn (p+7) u-PA^{-/-}, t-PA^{-/-}, and wild type mice were studied. The development and characterization of these mice has been described previously³⁰⁻³³. Each group consisted of 11 to 14 mice.

Mice were placed in a tightly sealed chamber under normobaric hypoxia (FiO₂ 10%), which was maintained by simultaneous inflow of room air (2 l/min) and nitrogen (2 l/min) through the chamber. The oxygen concentration was continuously measured using an oxygen sensor. The chamber was opened every 5 days for 10 minutes to clean the cages and replenish food and water. Genotypic identical normoxic control mice were maintained in identical conditions in room air (FiO₂ 21%).

Adult mice were studied after 28 days hypoxia or normoxia, whereas newborn mice (with their mother in the cage) were studied after 10 days. After this period, hemodynamic measurements were performed (adult mice only), a blood sample was collected for hematocrit measurement, and the heart and lungs were removed for morphometric and histological analysis.

Thoracic Aortic Banding

Mice are anaesthetized by intraperitoneal injection of pentobarbital sodium (60 mg/kg). They are weighed and the chest wall is shaved and prepared. In the supine position, endotracheal intubation is performed under direct laryngoscopy, and mice are ventilated with a small animal respirator (Harvard apparatus: tidal volume of 1.0 ml, rate 100 breaths/min). After a skin incision a sternotomy is performed. An operating microscope aids dissection. A small animal retractor exposes the basis of the heart and the thymus. The thymus is carefully dissected from the underlying aorta. The transverse aortic arch is ligated (7.0 Silk) between the innominate and left common carotid arteries with an overlying 27 gauge needle, and then the needle is removed, leaving a discrete region of stenosis. Sham operation consists of suture placed around the aorta without constriction. The chest cavity and skin are then closed using 5.0 silk. The mouse is removed from the expirator and once spontaneous respiration resumes, the endotracheal tube is withdrawn. The mice remain in a supervised setting on a warming pad, until fully conscious.

PAI-1 Gene Transfer.

The replication-deficient adenovirus expressing human PAI-1 (AdPAI-1) (Carmeliet et al. (1997), Circulation, 96, 3180) or control AdRR5 virus (Alcom et al. (1993) Mol. Endocrinol. 7, 1072) are purified to stocks of >10¹⁰ plaque forming units (pfu). Three days before constriction of the transverse aorta, 100 μl of 1.3×10⁹ pfu AdPAI-1 or control AdRR5 virus diluted in 0.9% NaCl is injected in the tail vein of WT mice. At five days after virus injection, PAI-1 plasma levels are measured using a murine monoclonal antibody based enzyme linked immunosorbent assay for PAI-1 in 100 μl blood, sampled from the retroorbital plexus. Virus-injected mice are analyzed at indicated times for morphology, histology and cardiac function.

Histology

Constricted or sham operated mice 1, 4, 7, 14 and 49 days after surgery are anaesthetized and perfused at physiological pressure via the abdominal aorta with a 0.9% NaCl solution until the blood is removed. After taking out the heart, left and right ventricle are dissected, blotted dry and weighted. Left ventricle is then directly cryo-embedded or first post-fixated in 1% para-formaldehyde, followed by cryo-embedding or embedding in paraffin. Six-μm thick sections are made for further histological analysis. All morphometric analysis and counting are performed using Quantimet 600. Mean myocyte area is evaluated in the subendocardial layer, the central and the sub-epicardial layer of the septum and the left ventricle, on haemalium-eosin stained sections. Collagen type-I and-III is stained using sirius red, and the amount of collagen is quantified as percentage sirius red staining area per total cardiac area.

Hemodynamic Measurements

For pressure measurements, mice are anesthetized with urethane (2.1 mg/g body weight, given subcutaneously; Sigma, Brussels, Belgium) and ventilated as described above. The skin above the thymus and trachea is opened. After dissection of the thymus, the right carotid artery is separated from the surrounding muscle, and ligated distally. After proximal ligation, a small incision is made in the right carotid artery. A 1.4 French high-fidelity catheter-tip micromanometer (SPR-671; Millar instruments, Houston, Tex.) is inserted through the right carotid artery and forwarded in the left ventricular cavity. The left ventricular pressure (Siemens Pressure Amplifier 863, Elema, Solna, Sweden) is amplified and unfiltered digitized using an analog-to-digital converter (Dataq DA Convert DI-205, DATAQ Instruments, Akron, Ohio) at a sampling rate of 2000 Hz. Digital files are recorded and analyzed with commercially available software (WinDaq Acquisition DI200AC version 1.65; WinDaq advanced CODAS analysis software, DATAQ Instruments, Akron, Ohio).

For M-mode echocardiography, animal are first lightly anesthetized with intraperitoneal ketamine 50 mg/kg and xylazine 2.5 mg/kg, and studied in conscious condition on a warming pad. 2D guided M-mode echocardiography in the mouse is performed with a 12-MHz transdcucer (Hewlett Packard) on a Hewlett Packard (HP 5000) echocardiograph. Views are taken in planes that approximates the parasternal short-axis view and in M-mode we measure intraventricular septum thickness, end-diastolic left ventricular internal diameter (EDD), end-systolic left ventricular internal diameter (ESD) and left ventricular posterior wall thickness, using leading edge-to-leading edge convention. Percent fractional shortening is then calculated.

The right ventricular systolic and diastolic pressures were measured in anesthetized mice (sodium pentobarbital, 60 mg/kg, i.p.) by transthoracic puncture as previously described³⁴. Right ventricular pressure was measured continuously for 5 minutes using a pressure transducer (Model AA 016, Baxter, Uden, the Netherlands), positioned at a height of 0.5 cm above the level of the sternum. Systemic arterial blood pressure was continuously measured over a 5 min period by insertion of the needle into the abdominal aorta. Hemodynamic measurements were displayed on an oscilloscope (Pressure Amplifier 863, Elema, Solna, Sweden) and analyzed on a PC-based computer program (Windaq Software vs 1.37, Dataq Instruments Inc, Akron, Ohio).

Blood Sampling and Hematocrit Measurement

Blood samples were collected from the abdominal aorta and anticoagulated with EDTA (10 mmol/l). Hematocrit was measured using an automated cell counter (Abbott Cell-Dyn 1330 system, Abbott Park, Ill.).

Measurement of Right Ventricular Hypertrophy

The right ventricular free wall was separated from the left ventricle and septum under a dissecting microscope, essentially according to the procedure of Fulton et al.³⁵ The right ventricle and the left ventricle/septum were dried at 90° for 48 hours and 72 hours. If the difference in weight between these two time intervals was greater than 0.5 mg, the specimens were dried for another 24 hours. Right ventricle and left ventricle plus septum were weighed separately. Results were expressed as ratio of right ventricle weight over left ventricle plus septum weight or right ventricle weight over bodyweight.

Morphometric Analysis

A cannula was introduced in the right atrium and mice were perfused with 1% phosphate buffered para-formaldehyde at 100 cm H₂O pressure for 5 minutes. Subsequently, the trachea was cannulated and 1% phosphate buffered para-formaldehyde was perfused at 30 cm H₂O through the airways, which resulted in a distension of the lungs and a smoothening of the pleural surface. The heart and lungs were removed en bloc and the heart was separated from the lungs and the large vessels. The samples were cryoembedded or postfixed for 24 hours in 1% phosphate buffered para-formaldehyde, washed in phosphate-buffered saline, dehydrated, embedded in paraffin, and sliced. Verhoeff's-van Gieson stains were performed on 4 μm sections. In addition, sections of the heart (7 μm) were used for sirius red staining and immunostaining of laminin, thrombomodulin, t-PA, u-PA, or matrix metalloproteinase-9 (MMP-9), as described previously^{36,37}. In situ zymographic activity of t-PA and u-PA was performed using gel overlays on 7 μm unfixed cryosections. T-PA- and u-PA-specific lysis was determined by addition of neutralizing antibodies specific for t-PA or u-PA to the fibrin gel³⁷. Morphometric analysis was performed using the Quantimet 600 image analysis system (Leica, Brussels, Belgium).

Hypoxia-induced pulmonary vascular remodeling was assessed by two different methods, as previously described³⁴. Firstly, the peripheral vessel density (defined as the number of vessels per 100 alveoli) was determined. To this effect, in each lung section 5×500 alveoli were counted. Peripheral arteries were defined as all vessels, landmarked to airway structures distal to the terminal bronchioli. Non-muscularized and partly or fully muscularized vessels were scored separately. Secondly, of 10 muscularized vessels in each section the thickness of the medial wall was measured and related to the external diameter of the vessel. Media thickness was determined by measuring the diameter between the internal and external elastic lamina. For each vessel the smallest external diameter (defined as the distance between the external elastic laminae) was measured. Results are expressed as ratio of media thickness over external vascular diameter (%). Morphometric analysis of remodeling of the right ventricle of the heart consisted of measurement of right ventricular myocyte hypertrophy, measurement of the number of subendocardial capillaries and assessment of the collagen contents of the right ventricle. Right ventricular myocyte hypertrophy was measured as the cross-sectional area of at least 50 individual cardiomyocytes per heart in the right ventricle on laminin-stained sections to delineate the basement membrane. The number of subendocardial capillaries were counted on thrombomodulin-stained sections (to visualize endothelial cells) and expressed as number of capillaries/mm². Collagen type I and IIII contents of the right ventricle was quantified on sirius red-stained sections.

Statistical Analysis

Results are presented as mean values±SD's. Statistical analysis was performed by ANOVA and subsequent Newman-Keuls test. A p-value <0.05 was considered statistically significant.

Tables

TABLE 1
Morphology after transverse aortic banding.

| | WT | u-PA^{-/-} | WT TAB | u-PA^{-/-} TAB | WT TAB | u-PA^{-/-} TAB |
|---|---|---|---|---|---|---|
| | sham | sham | 2 wk | 2 wk | 7 wk | 7 wk |
| LV/body | 3.7 ± 0.1 | 3.5 ± 0.2 | 5.3 ± 0.2* | 4.4 ± 0.4*# | 5.7 ± 0.3* | 4.3 ± 0.2*# |
| weight mg/g | | | | | | |
| LV Myocyte | 140 ± 10 | 138 ± 8 | 300 ± 10* | 190 ± 10*# | ND | ND |
| area, um² | | | | | | |
| % LV | 7 ± 1 | 6 ± 1 | 13 ± 1* | 8 ± 1*# | ND | ND |
| Collagen | | | | | | |
| content | | | | | | |

Sham indicates sham-operated normal mice;
TAB, transverse aortic banding studied after 2 and 7 weeks after operation;
LV, left ventricular;
WT, wild type mice.
n = 6 in sham-operated mice,
n = 6 to 9 in banded mice.
*p < 0.05 in TAB as compared to sham mice,
#p < 0.05 in u-PA^{-/-} as compared to WT mice.

TABLE 2
In vivo pressure measurements.

| | WT | u-PA^{-/-} | WT TAB | u-PA^{-/-} | WT TAB | u-PA^{-/-} |
|---|---|---|---|---|---|---|
| | sham | sham | 2 wk | TAB 2 wk | 7 wk | TAB 7 wk |
| LV systolic | 90 ± 4 | 80 ± 10 | 133 ± 4* | 144 ± 6* | 123 ± 8* | 136 ± 2* |
| pressure | | | | | | |
| Peak, +dP/dt, | 7800 ± | 8200 ± | 8100 ± | 11000 ± | 6100 ± | 10300 ± |
| +mmHgs/s | 630 | 850 | 460 | 890*# | 610* | 770*# |
| Peak, −dP/dt, | 5600 ± | 6100 ± | 6800 ± | 8700 ± | 5400 ± | 8700 ± |
| −mmHgs/s | 420 | 400 | 410 | 450* | 510 | 680*# |
| Heart rate | 560 ± | 540 ± 30 | 530 ± 10 | 550 ± 10 | 520 ± 20 | 530 ± 10 |
| | 20 | | | | | |

Sham indicates sham-operated normal mice;
TAB, transverse aortic banding studied after 2 and 7 weeks after operation;
LV, left ventricular;
WT, wild type mice.
n = 6 in sham-operated mice,
n = 6 to 9 in banded mice.
*p < 0.05 in TAB as compared to sham mice,
#p < 0.05 in u-PA^{-/-} as compared to WT mice after TAB.

TABLE 3
In vivo echocardiographic assessment.

| | | u-PA^{-/-} | WT TAB | u-PA^{-/-} TAB 2 |
|---|---|---|---|---|
| | WT sham | sham | 2 wk | wk |
| PW thickness, mm | 0.86 ± 0.1 | 0.84 ± | 1.5 ± 0.1* | 1.0 ± 0.05# |
| | | 0.2 | | |
| Septal wall thick- | 0.96 ± 0.1 | 0.98 ± | 1.6 ± 0.1* | 1.1 ± 0.1# |
| ness, mm | | 0.3 | | |
| LV diastolic | 4.2 ± 0.1 | 4.4 ± 0.2 | 4.7 ± 0.6 | 4.6 ± 0.7 |
| dimension, mm | | | | |
| LV systolic | 2.4 ± 0.1 | 2.6 ± 0.2 | 3.4 ± 0.7* | 2.9 ± 0.8# |
| dimension, mm | | | | |
| Fractional | 43 ± 2 | 42 ± 1 | 27 ± 5* | 48 ± 8# |
| shortening, % | | | | |

Sham indicates sham-operated normal mice;
TAB, transverse aortic banding studied after 2 weeks after operation;
LV, left ventricular;
PW, posterior wall;
WT, wild type mice.
n = 6 in sham-operated mice,
n = 6 to 9 in banded mice.
*p < 0.05 in TAB as compared to sham mice,
#p < 0.05 in u-PA^{-/-} as compared to WT mice after TAB.

TABLE 4
PAI-1 gene transfer.

| | | WT AdRR5, | WT AdPAI-1, |
|---|---|---|---|
| | WT AdRR5, | TAB | TAB |
| | sham | 2 wk | 2 wk |
| LV/body weight mg/g | 3.6 ± 0.1 | 5.1 ± 0.1* | 4.4 ± 0.2*# |
| LV systolic pressure | 86 ± 2 | 126 ± 4* | 137 ± 5*# |
| Peak, +dP/dt, | 8700 ± 520 | 9700 ± 430 | 11900 ± 450*# |
| +mmHgs/s | | | |
| Peak, −dP/dt, | 6200 ± 360 | 7200 ± 570 | 8900 ± 390*# |
| −mmHgs/s | | | |
| Heart rate | 540 ± 40 | 550 ± 10 | 570 ± 30 |

Sham indicates sham-operated mice;
TAB, transverse aortic banding studied 2 weeks after operation;
LV, left ventricular;
AdRR5, control replication deficient adenovirus;
AdPAI-1, replication deficient adenovirus carrying the humans PAI-1 gene;
WT, wild type mice.
n = 6 in sham-operated mice,
n = 9 to 11 in banded mice.
*p < 0.05 in TAB as compared to sham mice,
#p < 0.05 in AdPAI-1 as compared to AdRR5-treated mice after TAB.

TABLE 5
measurement of right ventricular pressure

| | RVSP | | MAP | hematocrit |
|---|---|---|---|---|
| | (mmHg) | RVDP (mmHg) | (mmHg) | (%) |
| wild type | | | | |
| 21% O₂ (n = 6) | 21 (±3.2) | 5.5 (±1.0) | 52 (±1.0) | 49 (±1.0) |
| 10% O₂ (n = 7) | 37 (±3.8) * | 15 (±3.7) * | 51 (±3.9) | 61 (±0.9) |
| u-PA -/- | | | | |
| 21% O₂ (n = 6) | 21 (±2.6) | 6.1 (±2.1) | 53 (±4.1) | 49 (±1.1) |
| 10% O₂ (n = 7) | 24 (±3.1) # | 8.8 (±2.8) # | 51 (±2.5) | 61 (±1.1) |
| u-PAR -/- | | | | |
| 21% O₂ (n = 7) | 20 (±4.0) | 6.1 (±1.5) | 49 (±2.9) | 48 (±1.2) |
| 10% O₂ (n = 7) | 27 (±2.5) *,# | 12 (±1.7) * | 53 (±3.2) | 61 (±0.9) |
| t-PA -/- | | | | |
| 21% O₂ (n = 6) | 21 (±2.2) | 5.8 (±1.2) | 51 (±3.3) | 48 (±1.0) |
| 10% O₂ (n = 8) | 34 (±3.1) * | 15 (±2.6) * | 51 (±2.8) | 62 (±1.4) |
| plg -/- | | | | |
| 21% O₂ (n = 7) | 21 (±2.3) | 7.2 (±1.3) | 54 (±4.1) | 49 (±1.5) |
| 10% O₂ (n = 8) | 24 (±1.3) # | 6.3 (±2.7) # | 58 (±3.6) | 60 (±1.7) |

Right ventricular systolic and diastolic pressure (RVSP and RVDP), mean arterial pressure (MAP), and hematocrit in mice with various genotypes under normoxic and hypoxic conditions. Mean values, (±SD) and statistical significance (p < 0.05) are given (*normoxic versus hypoxic, # genotype versus wild type).

TABLE 6
measurement of pulmonary vascular remodelling

| | vascular density | | | media |
|---|---|---|---|---|
| | non-muscularized | | partly/fully | thickness/ |
| | vessels | | muscularized vessels | vascular |
| | (arteries/100 alveoli) | | (arteries/100 alveoli) | diameter (%) |
| wild type | | | | |
| 21% | O₂ | 2.4 (±0.2) | 4.0 (±0.3) | 6.1 (±1.2) |
| (n = 6) | | 1.7 (±0.1) | 3.1 (±0.2) | 13 (±1.5) |
| 10% | O₂ | | | |
| (n = 7) | | | | |
| u-PA^{-/-} | | | | |
| 10% | O₂ | 2.5 (±0.2) * | 4.0 (±0.2) * | 7.3 (±1.4)** |
| (n = 7) | | | | |
| u-PAR^{-/-} | | | | |
| 10% | O₂ | 2.1 (±0.2) | 3.5 (±0.3) | 10 (±1.2)* |
| (n = 6) | | | | |
| t-PA^{-/-} | | | | |
| 10% | O₂ | 1.8 (±0.2) | 2.9 (±0.2) | 13 (±1.9) |
| (n = 7) | | | | |
| plg^{-/-} | | | | |
| 10% | O₂ | 2.7 (±0.1)* | 4.1 (±0.1)* | 6.3 (±1.5) ** |
| (n = 7) | | | | |

Parameters of pulmonary vascular remodelling in hypoxic adult mice with various genotypes. Values of normoxic wild type mice are shown as well. There are no differences between the various genotypes under normoxic circumstances. Mean values, (±SD) and statistical significant differences versus wild type mice under hypoxia (*: p < 0.05; **: p < 0.01) are given.

TABLE 7
measurement of right ventricular hypertrophy

| | wild type mice (n = 5) | u-PA^{-/-} mice (n = 5) |
|---|---|---|
| cross-sectional diameter | 21% O₂: 250 (±40) | 21% O₂: 240 (±47) |
| cardiomyocytes (μm²) | 10% O₂: 340 (±68) | 10% O₂: 260 (±35)* |
| collagen contents | 21% O₂: 7.8 (±1.8) | 21% O₂: 7.5 (±1.9) |
| right ventricle (%) | 10% O₂: 14 (±2.1) | 10% O₂: 9.2 (±2.2)* |
| capillaries in | 21% O₂: 5200 (±210) | 21% O₂: 5200 (±250) |
| subendocardium (/mm²) | 10% O₂: 4400 (±260) | 10% O₂: 5300 (±210) |

Parameters of right ventricular hypertrophy in wild type mice and u-PA^{-/-} mice under normoxic and hypoxic conditions. Mean values, (SD) and statistical significant differences versus wild type mice (*: p < 0.05) are given.

References

1. Dor Y, Keshet E. Ischemia-driven angiogenesis. Trends Cardiovasc Med 1997; 7:289-94.

2. Pfeifer M, Blumberg F C, Wolf K, Sandner P, Elsner D, Riegger G A, Kurtz A. Vascular remodeling and growth factor gene expression in the rat lung during hypoxia. Respir Physiol 1998; 111:201-212.

3. Li H, Elton T S, Chen Y F, Oparil S. Increased endothelin receptor gene expression in hypoxic rat lung. Am J Physiol 1994; 266:L553-560.

4. Markewitz B A, Kohan D E, Michael J R. Hypoxia decreases endothelin-1 synthesis by rat lung endothelial cells. Am J Physiol 1995; 269:L215-220.

5. Kourembanas S, Marsden P A, McQuillan L P, Faller D V. Hypoxia induces endothelin gene expression and secretion in cultured human endothelium. J Clin Invest 1991; 88:1054-7.

6. Riva C, Chauvin C, Pison C, Leverve X. Cellular physiology and molecular events in hypoxia-induced apoptosis. Anticancer Res 1998; 18:4729-36.

7. Carmeliet P, Dor Y, Herbert J M, Fukumara D, Brusselmans K, Dewerchin M, Neeman M, Bono F, Abramovitch R, Maxwell P, Koch C J, Ratcliffe P, Moons L, Jain R K, Collen D, Keshert E. Role of HIF-1α in hypoxia-mediated apoptosis, cell proliferation, and tumor angiogenesis. Nature 1998; 394:485-90.

8. Thompson B T, Steigman D M, Spence C L, Janssens S P, Hales C A. Chronic hypoxic pulmonary hypertension in the guinea pig: effect of three levels of hypoxia. J Appl Physiol 1993;74:916-21.

9. Janssens S P, Thompson B T, Spence C R, Hales C A. Polycythemia and vascular remodeling in chronic hypoxic pulmonary hypertension in guinea pigs. J Appl Physiol 1991; 71:2218-23.

10. Jones R, Reid L. Vascular remodeling in clinical and experimental pulmonary hypertensions. Portland Press Ltd, London, 1995, pp. 47-116.

11. Ie Noble F A, Stassen F R, Hacking W J, Struijker Boudier H A. Angiogenesis and hypertension. J Hypertens 1998; 16:1563-72.

12. Rabinovitch M. Pulmonary hypertension: updating a mysterious disease. Cardiovasc Res 1997; 34:268-72.

13. Huabin L I, Chen S-J, Chen Y-F, Meng Q C, Durand J, Oparil S, Elton T S. Enhanced endothelin-1 and endothelin receptor gene expression in chronic hypoxia. J Appl Physiol 1994; 77:1451-59.

14. Morell N W, Atochina E N, Morris K G, Danilov S M, Stenmark K R. Angiotensin converting enzyme expression is increased in small pulmonary arteries of rats with hypoxia-induced pulmonary hypertension. J Clin Invest 1995; 96:1823-33.

15. Chen S-J, Chen Y-F, Opgenort T J, Wessale J L, Meng Q C, Durand J, DiCarlo V S, Oparil S. The orally active nonpeptide endothelin A-receptor antagonist A-127722 prevents and reverses hypoxia-induced pulmonary hypertension and pulmonary vascular remodeling in Sprague-Dawley rats. J Cardiovasc Pharmacol 1997; 29:713-25.

16. Herget J, Pelouch V, Kolar F, Ostadal B. The inhibition of angiotensin converting enzyme attenuates the effect of chronic hypoxia on pulmonary blood vessels in the rat. Physiol Res 1996; 45:221-6.

17. Fagan K A, Fouty B W, Tyler R C, Morris K G Jr, Hepler L K, Sato K, Lecras T D, Abman S H, Weinberger H D, Huang P L, McMurtry I F, Rodman D M. The pulmonary circulation of homozygous and heterozygous eNOS-null mice is hyperresponsive to mild hypoxia. J Clin Invest 1999; 103:291-9.

18. Janssens S P, Bloch K D, Nong Z, Gerard R D, Zoldhelyi P, Collen D. Adenoviral-mediated transfer of the human endothelial nitric oxide synthase gene reduces acute hypoxic pulmonary vasoconstriction in rats. J Clin Invest 1996; 98:317-24.

19. Powell P P, Klagsbrun M, Abraham J A, Jones R C. Eosinophils expressing heparin-binding EGF-like growth factor mRNA localize around lung microvessels in pulmonary hypertension. Am J Pathol 1993; 143:784-93.

20. Christou H, Yoshida A, Arthur V, Morita T, Kourembanas S. Increased vascular endothelial growth factor production in the lungs of rats with hypoxia-induced pulmonary hypertension. Am J Respir Cell Mol Biol 1998; 18:768-76.

21. Kourembanas S, Hannan R L, Faller D V. Oxygen tension regulates the expression of platelet-derived growth factor-B chain in human endothelial cells. J Clin Invest 1990; 86:6704.

22. Maruyama K, Ye C, Woo M, Venkatacharya H, Lines L D, Silver M M, Rabinovitch M. Chronic hypoxic pulmonary hypertension in rats and increased elastolytic activity. Am J Physiol 1991; 261:H1716-26.

23. Thompson K, Rabinovitch M. Exogenous leucocyte and endogenous elastases can mediate mitogenic activity in pulmonary artery smooth muscle cells by release of extracellular matrix-bound basic fibroblast growth factor. J Cell Physiol 1996; 166:495-505.

24. Pinsky D J, Liao H, Lawson C A, Yan S F, Chen J, Carmeliet P, Loskutoff D J, Stern D M. Hypoxia-mediated suppression of fibrinolysis enhances pulmonary vascular fibrin deposition. J Clin Invest 1998; 102:919-28.

25. Graham C H, Fitzpatrick T E, McCrae K R. Hypoxia stimulates urokinase receptor expression through a heme protein-dependent pathway. Blood 1998; 91:3300-7.

26. Bansal D D, Klein R M, Hausmann E H, MacGregor R R. Secretion of cardiac plasminogen activator during hypoxia-induced right ventricular hypertrophy. J Mol Cell Cardiol 1997; 29:3105-14.

27. Bloor C M, Nimmo L, McKirnan M D, Zhang Y, White F C. Increased gene expression of plasminogen activators and inhibitors in left ventricular hypertrophy. Mol Cell Biochem 1997; 176:265-71.

28. Saksela O, Rifkin D B. Release of basic fibroblast growth factor-heparan sulfate complexes from endothelial cells by plasminogen activator-mediated proteolyitc activity. J Cell Biol 1990; 110:767-775.

29. Taipale J, Koli K, Keski-Oja J. Release of transforming growth factor-β1 from the pericellular matrix of cultured fibroblasts and fibrosarcoma cells by plasmin and thrombin. J Biol Chem 1992; 267:25378-84.

30. Carmeliet P, Moons L, Collen D. Mouse models of angiogenesis, arterial stenosis, atherosclerosis and hemostasis. Cardiovasc Res 1998; 39:8-33.

31. Carmeliet P, Schoonjans L, Kieckens L, Ream B, Degen J, Bronson R, de Vos R, van den Oord J J,, Collen D, Mulligan R C. Physiological consequences of loss of plasminogen activator gene function in mice. Nature 1994; 368:419-24.

32. Ploplis V A, Carmeliet P, Vazirzadeh S, van Vlaenderen I, Moons L, Plow E F, Collen D. Effects of disruption of the plasminogen gene on thrombosis, growth, and health in mice. Circulation 1995; 92:2585-93.

33. Dewerchin M, van Nuffelen A, Wallays G, BouchéA, Moons L, Carmeliet P, Mulligan R C, Collen D. Generation and characterization of urokinase receptor-deficient mice. J Clin Invest 1996; 97:870-8.

34. Hales C A, Kradin R L, Brandstetter R D, Zhu Y J. Impairment of hypoxic pulmonary artery remodeling by heparin in mice. Am Rev Respir Dis 1983; 128:747-51.

35. Fulton R M, Hutchinson E C, Jones M. Ventricular weight in cardiac hypertrophy. Br Heart J 1952; 14:413-20.

36. Carmeliet P, Moons L, Lijnen R, Baes M, Lemaitre V, Tipping P, Drew A, Eeckhout Y, Shapiro S, Lupu F, Collen D. Urokinase-generated plasmin activates matrix metalloproteinases during aneurysm formation. Nature Genet 1997; 17:439-44.

37. Carmeliet P, Moons L, Herbert J M, Crawley J, Lupu F, Lijnen R, Collen D. Urokinase-type but not tissue-type plaminogen activator mediates arterial neointima formation in mice. Circ Res 1997; 81:829-39.

38. Heymans S, Luttun A, Nuyens D, Theilmeier G, Creemers E, Moons L, Dyspersin G D, Cleutjens J P, Shipley M, Angellilo A, Levi M, Nube O, Baker A, Keshet E, Lupu F, Herbert J M, Smits J F, Shapiro S D, Baes M, Borgers M, Collen D, Daemen M J, and Carmeliet P. Inhibition of plasminogen activators or matrix metalloproteinases prevents cardiac rupture but impairs therapeutic angiogenesis and causes cardiac failure. Nature Medicine 1999; 5:1135-42.

39. Chang A W, Kuo A, Barnathan E S, Okada S S. Urokinase-dependent upregulation of smooth muscle cell adhesion to vitronectin by urokinase. Arterioscler Thromb Vasc Biol 1998; 18:1855-60.

40. Hunter C, Barer G R, Shaw J W, Clegg J. Growth of the heart and lungs in the hypoxic rodents: a model for human hypoxic disease. Clin Sci 1974; 46:375-91.

1. Use of urokinase inhibitors for the manufacture of a medicament for the treatment and/or prophylaxis of pulmonary hypertension.

2. Use of urokinase inhibitors for the manufacture of a medicament for the treatment and/or prophylaxis of cardiac remodelling

3. A pharmaceutical composition comprising urokinase inhibitors for the treatments according to claims 1 and 2.

4. Use of plasminogen activator inhibitors and functional fragments thereof for gene therapy of pulmonary hypertension and/or cardiac remodelling.
